(19) United States
(12) Patent Application Publication
(10) Pub. No.: US 2023/0149430 A1
(43) Pub. Date: May 18, 2023

(54) COMPOSITIONS AND METHODS FOR THE TREATMENT OF INFECTION-INDUCED CARDIOMYOPATHY

(71) Applicant: Duke University, Durham, NC (US)

(72) Inventor: Kenneth W. Lyles, Durham, NC (US)

(21) Appl. No.: 17/907,681

(22) PCT Filed: Apr. 5, 2021
(86) PCT No.: PCT/US2021/025712

§ 371 Date: Sep. 29, 2022

Related U.S. Application Data
(60) Provisional application No. 63/005,521, filed on Apr. 6, 2020.

(51) Int. Cl. A61K 31/663; A61K 31/59; A61K 45/06; A61P 29/00; A61P 31/14

(52) U.S. Cl. CPC A61K 31/663; A61K 31/59; A61K 45/06; A61P 29/00; A61P 31/14

(57) ABSTRACT

The present invention provides a composition and method of treating infection-induced cardiomyopathy in a subject, the method comprising administering to the subject a therapeutically effective amount of a nitrogen-containing bisphosphonate or a pharmaceutically effective salt thereof, wherein the nitrogen-containing bisphosphonate is useful as a therapeutic agent, in particular in the treatment, therapy or prophylaxis of the novel coronavirus COVID-19.

### CROSS-REFERENCE TO RELATED APPLICATIONS

The present application claims priority to U.S. Provisional Patent Application Ser. No. 63/005,521, filed on Apr. 6, 2020, the contents of all are incorporated herein by reference in their entirety.

### BACKGROUND OF THE INVENTION

### Technical Field

This invention relates to the use of nitrogen-containing bisphosphonates (NCB) or a pharmaceutically effective salts thereof as effective therapies to treat infection-induced, particular viral-induced (i.e., COVID-19) cardiomyopathies.

### Related Art

Cardiomyopathy is a general term used to describe a diverse group of diseases of the heart muscle. For most people with cardiomyopathy, their hearts don't function normally because the heart has become either enlarged, abnormally thick, abnormally rigid, or unable to transmit electrical impulses in a normal fashion. These changes in the heart muscle correspond to the four principal types of cardiomyopathy-dilated cardiomyopathy, hypertrophic cardiomyopathy, restrictive cardiomyopathy, and arrhythmogenic right ventricular cardiomyopathy. Included in each of these categories are several dozen types of cardiomyopathy that are distinguished from one another containing upon their individual cause.

A wide variety of infectious diseases can result in cardiomyopathy, including those caused by viruses, bacteria, chlamydia, rickettsia, fungi, and protozoa. In light of the current pandemic, evidence is mounting showing that certain coronaviruses, in particular SARS-CoV-2 which causes COVID-19, can cause acute myocarditis through a number of suspected mechanisms, including but not limited to, ACE2-mediated direct damage, hypoxia-induced myocardial injury, increased levels of D-dimers causing coagulopathy, cardiac microvascular damage, and systemic inflammatory response syndrome. (1)

Recent data suggests that human pathogenic coronaviruses (severe acute respiratory syndrome coronavirus [SARS-CoV] and SARSCoV-2 (COVID-19)) bind to their target cells through angiotensin-converting enzyme 2 (ACE2), which is expressed by epithelial cells of the lung (Type 11, alveolar cells) intestine, kidney, and blood vessels. Angiotensin-converting enzyme 2, highly expressed in the respiratory system, has been identified as a functional receptor for severe acute respiratory syndrome coronavirus-2. Notably and importantly, ACE2 is also expressed in the cardiovascular system, and there are multiple cardiovascular implications of COVID-19. Recently, cardiovascular risk factors and cardiovascular disease have been associated with severe manifestations and poor prognosis in patients with COVID-19. More important, patients with COVID-19 may have thrombotic and coagulation abnormalities, promoting a hypercoagulable state and resulting in an increased rate of thrombotic and thromboembolic events. (2)

The expression of ACE2 is substantially increased in patients with type 1 or type 2 diabetes and cardiac disfunction who are treated with ACE inhibitors and angiotensin II type-I receptor blockers (ARBs). This likely explains why patients above age 65 who are usually associated with these co morbidities are more susceptible to this disease.

Once the lung is infected, the virus uses RNA polymerase to replicate in the alveolar cells and use the cellular machinery to translate the RNA to proteins. Finally, multiple viral particles are produced and released. This causes reinfection and the viruses can be transmitted to other people. Further it is known that COVID-19 can stimulate a cytokine storm in the lungs, such as an increase of IL-2, IL-6, IL-7, GSCF, IP10, MCP1, MIP1A, and TNFα, followed by the edema, dysfunction of the air exchange, acute respiratory distress syndrome, acute cardiac injury and a secondary infection which may lead to death.

The World Health Organization (WHO) has reported that almost 80% of the cases have mild to moderate infection (Classified as cough and fever but not requiring hospitalization) 13.8% have severe and 6.1% have critical disease. The median incubation period of the infection is about 4-5 days before symptom onset, with 97.5% of symptomatic patients developing symptoms within 11.5 days. At hospitalization, patients with COVID-19 exhibit a fever, dry cough and sometimes dyspnea. Within 5-6 days of symptom onset, SARS-CoV-2 viral load reaches its peak and in severe cases the infection progresses to acute respiratory distress syndrome (ARDS), around 8-9 days after symptom onset.

The absence of successful treatments to reduce the negative effects of COVID-19 poses a severe global health threat due to its pandemic determination. To date, there is no prophylactic or therapeutic solution to efficiently treat the negative effects of this epidemic respiratory viral pathogen that has reached a pandemic level. Due to the highly infectious nature of this disease and the mounting death toll, there is an urgent need for the development of effective therapies to treat infection-induced, particularly viral-induced (i.e., COVID-19) cardiomyopathies.

### SUMMARY OF THE INVENTION

The present invention provides a composition and method of treating infection-induced cardiomyopathy in a subject, the method comprising administering to the subject a therapeutically effective amount of a nitrogen-containing bisphosphonate or a pharmaceutically effective salt thereof, wherein the nitrogen-containing bisphosphonate is useful as a therapeutic agent in the treatment, therapy or prophylaxis of coronavirus COVID-19 and variants thereof, wherein the nitrogen-containing bisphosphonate has the effect of reducing ACE2-mediated direct damage, hypoxia-induced myocardial injury, levels of D-dimers, cardiac microvascular damage, and/or cytokine release syndrome in the subject, thereby reducing mortality rate and comorbidity effects of the coronavirus COVID-19 and variants thereof.

In one aspect, the present invention provides a method of treating, reducing the negative effects or preventing infection-induced cardiomyopathy in a subject, the method comprising, administering to the subject a therapeutically effective amount of a nitrogen-containing bisphosphonate or a pharmaceutically effective salt thereof,

In the present invention, the nitrogen-containing bisphosphonate is selected from the group consisting of pamidronate (APD, Aredia), neridronate (Nerixia), olpadronate, alendronate (Fosamax), ibandronate (Boniva), risedronate (Actonel), zoledronic acid (Zometa, Aclasta), cimadronate, clodronate, tiludronate, etidronate, piridronate, and/or functional derivatives and/or analogues and/or esters, and/or salts thereof. In certain embodiments, the preferred nitrogen-containing bisphosphonate comprises zoledronic acid.

The cardiomyopathy addressed in the present invention is due to an infection selected from the group consisting of a viral infection, a yeast infection, a bacterial infection, a parasitic infection, a fungal infection, and combinations thereof. The viral infection comprises an infection caused by a coronavirus. In some embodiments, the viral infection comprises SARS, MERS, COVID-19 and variants thereof. In certain embodiments, the viral infection comprises COVID-19 and variants thereof.

In some methods, the dosage of nitrogen-containing bisphosphonate is from about 30 μg/kg to about 125 μg/kg. The nitrogen-containing bisphosphonate is administered prior to the infection, at the time of infection or after the infection.

Another aspect of the present disclosure provides a method of treating, reducing the negative effects or preventing infection-induced cardiomyopathy in a subject exposed to COVID-19 or variants thereof, the method comprising, consisting of, or consisting essentially of administering to the subject a therapeutically effective amount of zoledronic acid or a pharmaceutically effective salt thereof to reduce or prevent the development of new cardiac pathologies and/or exacerbation of underlying cardiovascular disease in the infected subject.

Administration of zoledronic acid can occur prior to the manifestation of symptoms of the COVID-19 virus and variants thereof, such that the virus is prevented or alternatively, delayed in its progression. The prophylactic methods of the present invention can be carried out in a similar manner to the therapeutic methods described herein, although dosage and treatment regimens may differ.

This present invention includes treatment or therapy of patients infected with COVID-19 and variants thereof including subjects with symptomatic or asymptomatic COVID-19 infections and variants thereof.

The nitrogen-containing bisphosphonate of the present invention is administered in a manner compatible with the dosage formulation, and in such amount as being therapeutically effective. The quantity to be administered depends on the subject to be treated, capacity of the subject's immune system to generate a cellular immune response, and degree of protection desired. Precise amounts of active ingredient required to be administered depend on the judgment of the practitioner and are peculiar to each individual.

In addition, because the use of more than one active agent may provide a better therapeutic method, and this is particularly true when the different agents act by different mechanisms, this invention also includes therapeutic compositions including both the nitrogen-containing bisphosphonate (NCB) according to the present invention together at least one other therapeutic agent(s) selected from the group consisting of an anti-viral agent, an anti-fungal agent, an anti-bacterial agent, an anti-yeast agent, an anti-parasitic agent, an anti-protozoal agent, an anti-inflammatory agent (e.g., NSAIDS, Cox-2 inhibitors, corticosteroids, etc.), and combinations thereof. In one embodiment, the other therapeutic agent comprises an anti-viral agent. In some embodiments, the anti-viral agent is selected from the group consisting of chloroquine, hydroxychloroquine, remdesivir (Gilead Sciences), favilavir (National Medical Products Administration of China), fusogenix (EntosPharmaceuticals), ChAd0x1 (Univ. of Oxford), Gimsilumab (Roivant Sciences), AdCOVID (Altimmune), TJM2 (1-Mab Biopharma), AT-100 (Airway Therapeutics), TZLS-501 (Tiziana Life Sciences), OYA1 (OyaGen), BPI-002 (BeyondSpring), INO-4800 (Inovio Pharmaceuticals), NP-120 (Algernon Pharmaceuticals), APNO1 (APEIRON Biologics), mRNA-1273 (Moderna), Avian Coronavirus Infectious Bronchitis Virus (IBV) vaccine (MIGAL Research Institute), TNX-800 (Tonix Pharmaceuticals), Btilacidin (Innovation Pharmaceuticals), Recombinant subunit vaccine (Clover Biopharmaceuticals), leronlimab (CytoDyn), Linear DNA Vaccine (Applied DNA Sciences), BXT-25 (BIOXYTRAN), INO-4700 (Inovio), Actemra (Roche), Galidesiver (Biocryst Pharma), REGN3048-3051 (Regeneron), SNG001 (Synairgen Research), Aminoboost (lattice Biologics), and the like.

Administration of the at least one other therapeutic agent(s) maybe administered prior to the nitrogen-containing bisphosphonate, at the time of nitrogen-containing bisphosphonate or is administered after the nitrogen-containing bisphosphonate.

In another aspect, the present invention further comprises administering to the subject a therapeutically effective amount of Vitamin D in combination with the NCB, which can be administered before, simultaneously or after the dosage of Vitamin D.

In another embodiment, the nitrogen-containing bisphosphonate is administered transdermally, orally, or intravenously.

Another aspect of the present disclosure provides a composition comprising a nitrogen-containing bisphosphonate in a therapeutically effect amount to treat an infection-induced cardiomyopathy in a subject, the composition is formulated to deliver from about 30 μg/kg to about 125 μg/kg of the nitrogen-containing bisphosphonate to the subject.

In another aspect, the present invention provides a method for alleviating the myocardial cytopathic destructive effects of COVID-19 infection in a human patient comprising administering to said patient a therapeutic amount of Zoledronic acid according to the present invention. The therapeutic amount of zoledronic acid comprises about 30 μg/kg to about 125 μg/kg of the nitrogen-containing bisphosphonate to the subject, and particularly about 100 μg/kg.

Another aspect of the present disclosure provides a pharmaceutical composition for the treatment of an infection-induced cardiomyopathy in a subject, the composition comprising a nitrogen-containing bisphosphonate in a therapeutically effect amount to treat an infection-induced cardiomyopathy in a subject, the composition is formulated to deliver from about 30 μg/kg to about 125 μg/kg of the nitrogen-containing bisphosphonate to the subject and a pharmaceutically acceptable carrier.

In a still further aspect, the present invention provides for a kit for treating a subject against COVID-19 and variants thereof wherein the kit comprises a therapeutically effective amount of a nitrogen-containing bisphosphonate and may further comprise instructions for use.

In yet another aspect, the present invention provides for the use of a therapeutically effective amount of a nitrogen-containing bisphosphonate in a medicament or in the manufacture of a medicament for the treatment of the negative effects due to COVID-19 and variants thereof.

These and other advantages and features of the present invention will be described more fully in a detailed description of the preferred embodiments which follows.

### DETAILED DESCRIPTION OF THE INVENTION

Cardiovascular disease (CVD) is the most common co-morbidity associated with COVID-19 and the fatality rate in COVID-19 patients with CVD is higher compared to other co-morbidities. Autopsies of COVID-19 patients reveal an infiltration of inflammatory mononuclear cells in the myocardium, confirming the role of the cytokine storm in response to COVID-19 infection. Cytokines are inflammatory immunologic proteins that are in the body to fight off infections but too many can result in an immune system gone wild. The body's own killer immune cells, in such an increased amount, can lead to organ failure and death. Currently, respiratory failure from acute respiratory distress syndrome (ARDS) is the leading cause of mortality of patients with COVID-19 and variants thereof and also suggesting potential causal mechanisms for the development of new cardiac pathologies and/or increasing damage of underlying CVDs in infected patients. Thus, the present invention provides for a novel approach to reduce the negative effects caused by direct cardiotoxicity through infecting the cells (cardiomyocytes).

The use of a NCB, and preferably zoledronic acid, as described in the present invention, rapidly alleviates the symptoms and signs related to COVID-19 and variants thereof. The negative effects of COVID-19 include acute myocarditis through a number of suspected mechanisms, including but not limited to, ACE2-mediated direct damage, hypoxia-induced myocardial injury, increased levels of D-dimers causing coagulopathy, cardiac microvascular damage, and cytokine release syndrome in treated patients. The reduction of these symptoms results in a substantial clinical improvement of critically ill patients, allows for a better management of the patient by the intensive care unit (ICU) team and most important reduces mortality of treated patients. A particular advantage of the proposed treatment of using a NCB is that it does not induce immunodeficiency in the patient, unlike other conventional therapies containing on steroids or other immunosuppressants. The preservation of a certain degree of immunocompetence in treated patients reduces the possibility of emergencies due to other opportunistic infections that are very common in intensive care.

Further the present invention provides for a composition and method using a nitrogen-containing bisphosphonate to reduce levels of D-dimers in a treated subject. Some important markers of organ dysfunction and coagulopathy found in subjects with Covid-19 includes higher levels of D-dimers. D-dimers are protein products of cross-linked fibrin degradation that are present in the blood of most healthy individuals but have been found in dangerous levels in subjects experiencing the effects of COVID-19 and other respiratory viral infections, including viral pneumonia due to influenza. Thus, the reduction of D-dimers is another advantageous effect of the present invention.

Still another indicator to be reviewed and analyzed is the level of troponins that are a group of proteins found in heart (cardiac) muscle fibers that regulate muscular contraction. Normally, troponin is present in very small quantities in the blood. However, when there is damage to heart muscle cells, troponin is released into the blood. The more damage there is, the greater the concentration in the blood. (3) As such, measuring the level of cardiac-specific troponin in the blood can help detect heart injury.

To date, there are no previous reports on the use of NCB against virus infections especially due to COVID-19, and particularly the capacity of zoledronic acid to reduce and/or inhibit the negative effects relating to myocardial injury and without causing immunodeficiency. Zoledronic acid has been previously used to provide advantageous effects to subjects with heart disease (4). There is further discuss of such treatments described in U.S. Pat. Nos. 9,867,838 and 9,949,992, the contents of which are included herein by reference.

However, in those previously discussed methods the subjects were not experiencing the negative effects of COVID 19 or a variant thereof, wherein the negative effects causing such myocardial injuries are due to acute respiratory distress syndrome and cytokines storms. As such, the present invention provides for a novel use of NCBs to address viral infections and the negative effects of myocardial injuries thereof.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations when interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

The term “bisphosphonate,” as used herein, means any compound which is an analog of endogenous pyrophosphate whereby the central oxygen is replaced by carbon. Bisphosphonates include aminobisphosphonates and nitrogen-containing bisphosphonates. Suitable bisphosphonates include but are not limited to the following compounds: pamidronate (APD, Aredia), neridronate (Nerixia), olpadronate, alendronate (Fosamax), ibandronate (Boniva), risedronate (Actonel), zoledronic acid (Zometa, Aclasta), cimadronate, clodronate, tiludronate, etidronate, piridronate, and/or functional derivatives and/or analogues and/or esters, and/or salts thereof. In some embodiments, the nitrogen-containing bisphosphonate is selected from the group consisting of zoledronic acid, alendronate, risedronate, and ibandronate, and derivatives, esters, and salts thereof. In certain embodiments, the nitrogen-containing bisphosphonate comprises zoledronic acid.

The term “zoledronic acid,” as used herein, means to include the free acid itself, i.e., 1-hydroxy-2-(imidazol-1-yl)ethane-1,1-diphosphonic acid, as well as any pharmaceutically acceptable salts and hydrates thereof and solvates thereof formed from other solvents used for its crystallization. 1-hydroxy-2-(imidazol-1-yl)ethane-1,1-diphosphonic acid and its pharmacologically acceptable salts, hydrates and solvates are well-known from the literature. They can be prepared by procedures known in the art, such as described, e.g., in U.S. Pat. No. 4,939,130. See also U.S. Pat. Nos. 4,777,163 and 4,687,767. The contents of the latter three patents are hereby incorporated by reference in their entirety.

The term “cardiomyopathy” refers to those diseases of the heart muscle that make it harder for the heart to pump blood to the rest of the body. Cardiomyopathy as defined herein also includes myocarditis, also known as inflammatory cardiomyopathy. The term “infection-induced cardiomyopathy” refers to those diseases of the heart caused as the result of an infection. As used herein, the term “infection” refers to the invasion and multiplication of microorganisms, such as bacteria, viruses, parasites, protozoa, fungi, yeast, and the like in the body of a subject.

In some embodiments, the infection causes cardiomyopathy in the subject. In some embodiments, the cardiomyopathy is due to an infection selected from the group consisting of a viral infection, a yeast infection, a bacterial infection, a parasitic infection, a fungal infection, and combinations thereof. In some embodiments, the infection comprises a viral infection. In other embodiments, the viral infection comprises an infection caused by a coronavirus. In some embodiments, the viral infection comprises SARS, MERS, COVID-19 and variants thereof. In certain embodiments, the viral infection comprises COVID-19 or variants thereof.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “preventing” means minimizing or partially or completely inhibiting the development of infection-induced cardiomyopathy in a mammal at risk for developing infection-induced cardiomyopathy. Determination of whether infection-induced cardiomyopathy is minimized or prevented by administration of a bisphosphonate is made by known methods.

The term “effective amount” or “therapeutically effective amount” means an amount of a compound or combination of compounds that ameliorates, attenuates, or eliminates one or more symptoms of infection-induced cardiomyopathy or prevents or delays the onset of one or more symptoms of infection-induced cardiomyopathy as defined herein.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In certain embodiments, the subject is a human suffering from infection-induced cardiomyopathy.

The compositions (e.g., nitrogen-containing bisphosphonate) of the present disclosure may also be in the form of a pharmaceutical composition. The term “pharmaceutically acceptable,” as used herein, means that the carrier, diluent, excipients, and/or salt must be compatible with the other ingredients of the formulation, and not deleterious to the patient.

Examples of pharmaceutically acceptable salts of the compounds include salts derived from an appropriate base, such as an alkali metal (for example, sodium, potassium), an alkaline earth metal (for example, calcium, magnesium), ammonium and NR′₄⁺(wherein R′ is C₁-C₄ alkyl). Pharmaceutically acceptable salts of an amino group include salts of organic carboxylic acids such as acetic, lactic, tartaric, malic, lactobionic, fumaric, and succinic acids; organic sulfonic acids such as methanesulfonic, ethanesulfonic, isethionic, benzenesulfonic and p-toluenesulfonic acids; and inorganic acids such as hydrochloric, hydrobromic, sulfuric, phosphoric and sulfamic acids. Pharmaceutically acceptable salts of a compound having a hydroxyl group consist of the anion of said compound in combination with a suitable cation such as Na⁺, NH₄⁺, or NR′₄⁺(wherein R′ is for example a C₁0.4 alkyl group).

The term “a form of Vitamin D,” as used herein, means any form of Vitamin D and functionally active analogue including Vitamin D2 (ergocalciferol or calciferol) and Vitamin D3 (cholecalciferol); hormones including calcidiol, dihydrotachysterol and calcitriol; Vitamin D analogues or metabolites including doxercalciferol and paricalcitol.

Variants of the COVID-19 may include changes in the amino acids found in the spike protein of the virus, but also in the ORF protein region. For example, the B.1.1.7 variant has a notable mutation N501 Y. Other mutations have been found to have more infective including A222V, E484K, S477N and K417N/T. As the virus spreads and more people are infected additional variants are inevitable

The term “functionally active analog,” as used herein, means compounds derived from a particular parent compound by straightforward substitutions that do not result in a substantial (i.e., more than 100x) loss in the biological activity of the parent compound, where such substitutions are modifications well-known to those skilled in the art, e.g., esterification, replacement of hydrogen by halogen, replacement of alkoxy by alkyl, replacement of alkyl by alkoxy, etc.

The bisphosphonate (e.g., a nitrogen-containing bisphosphonate such as zoledronic acid), is preferably used in the form of pharmaceutical compositions that contain a therapeutically effective amount of the bisphosphonate active ingredient optionally together with or in admixture with inorganic or organic, solid or liquid, pharmaceutically acceptable carriers which are suitable for administration.

The pharmaceutical compositions may be, for example, compositions for enteral, such as oral, rectal, aerosol inhalation or nasal administration, compositions for parenteral, such as intravenous or subcutaneous administration, or compositions for transdermal administration, e.g., passive or iontophoretic. Preferably, the pharmaceutical compositions are for intravenous administration. The pharmaceutical compositions may also be for direct intracoronary injection or elution from an intravascular or intracardiac device.

The particular mode of administration and the dosage may be selected by the attending physician taking into account the particulars of the patient, especially age, weight, lifestyle, activity level, hormonal status, e.g., post-menopausal, and bone mineral density as appropriate. Most preferably, however, zoledronic acid is administered intravenously and the dosage of the zoledronic acid may depend on various factors, including sex, age, weight and/or individual condition of the subject.

Normally the dosage is such that a single dose of a nitrogen-containing bisphosphonate such as zoledronic acid or salt or hydrate thereof, is from about 0.002 to about 20.0 mg/kg, preferably from 0.01 to 1 mg/kg, and more preferably from about 0.04 mg/kg to about 0.125 mg/kg. The term “mg/kg” means mg of drug per kg body weight of the subject. The dosage will be determined to correspond with the frequency of administering the compound.

In accordance with the present disclosure, the bisphosphonate is dosed at intervals of at least about once a month, every three months, six months, e.g., once every 180 days, or less frequently, conveniently once a year, or at any interval in between, e.g., once every 7, 8, 9, 10 or 11 months. The dose mentioned above, either administered as a single dose or in several partial doses, is preferably administered once per year (understanding, of course, that it may not be exactly one year to date but rather at yearly check-ups).

Formulations in single dose unit form contain preferably from about 1% to about 90%, and formulations not in single dose unit form contain preferably from about 0.1% to about 20% of the zoledronic acid active ingredient. Pharmaceutical preparations for enteral and parenteral administration are, for example, those in dosage unit forms, such as drages, tablets or capsules and also ampoules. They are prepared in a manner known per se, for example, by means of conventional mixing, granulating, confectioning, dissolving or lyophilizing processes.

For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or drage cores. Suitable carriers are especially fillers, such as sugars, for example, lactose, saccharose, mannitol or sorbitol, cellulose preparations and/or calcium phosphates, for example, tricalcium phosphate or calcium hydrogen phosphate, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, cross-linked polyvinylpyrrolidone, agar or alginic acid or a salt thereof, such as sodium alginate. Adjuncts are especially flow-regulating agents and lubricants, for example, silicic acid, talc, stearic acid or salts thereof, such as magnesium or calcium stearate, and/or polyethylene glycol. Drage cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as acetylcellulose phthalate or hydroxypropylmethylcellulose phthalate. Coloring substances or pigments may be added to the tablets or drage coatings, for example for the purpose of identification or to indicate different doses of active ingredient.

Other orally administrable pharmaceutical preparations are dry-filled capsules made of gelatin, and also soft, sealed capsules made of gelatin and a plasticizer, such as glycerol or sorbitol. The dry-filled capsules may contain the active ingredient in the form of a granulate, for example, in admixture with fillers, such as lactose; binders, such as starches; and/or glidants, such as talc or magnesium stearate, and, where appropriate, stabilizers. In soft capsules, the active ingredient is preferably dissolved or suspended in suitable liquids, such as fatty oils, paraffin oil or liquid polyethylene glycols, it being possible also for stabilizers to be added.

Parenteral formulations are especially injectable fluids that are effective in various manners, such as intra-arterially, intramuscularly, intraperitoneally, intranasally, intradermally, subcutaneously or preferably intravenously. Such fluids are preferably isotonic aqueous solutions or suspensions which can be prepared before use, for example, from lyophilized preparations which contain the active ingredient alone or together with a pharmaceutically acceptable carrier. The pharmaceutical preparations may be sterilized and/or contain adjuncts, for example preservatives, stabilizers, wetting agents and/or emulsifiers, solubilizers, salts for regulating the osmotic pressure and/or buffers.

Suitable formulations for transdermal application include an effective amount of the zoledronic acid active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time and means to secure the device to the skin.

In another embodiment, the present disclosure relates to ensuring that the subject has an adequate level of Vitamin D before the administration of the bisphosphonate compound and specifically zoledronic acid. The level of Vitamin D can be easy determined by a simple blood test that determines the level of Calcidiol (25-hydroxyvitamin D). The unit dose of Vitamin D will be determined by the specific form, the number of days of administration, age and condition of patient, and level of determined Vitamin D deficiency. For example, cholecalciferol may in a unit tablet dose of from about 400 to 5000 IU or in intramuscular form from about 50,000 units/cc to 100,000 units/cc; egocalciferol in unit capsule dose of from about 400 to 50,000 IU; oral calcitriol in a dose from about 0.10 to about 1 mcg which can be administered at least once a day or in multiple administrations; calcidiol or doxercalciferol, both of which are vitamin D analogues may be administered in dose units of from about 300 to 2000 IU.

In yet another embodiment, the present disclosure relates to a formulation that includes a bisphosphonate (e.g., a nitrogen-containing bisphosphonate such as zoledronic acid), a form of Vitamin D and optionally calcium in an essentially homogeneous mixture, wherein a solution or solid unit dose can be administered in a single dose. The single dose can be administered daily, monthly or yearly, or at some intermediate interval depending on the bisphosphonate compound.

In some embodiments, the nitrogen-containing bisphosphonate is selected from the group consisting of pamidronate (APD, Aredia), neridronate (Nerixia), olpadronate, alendronate (Fosamax), ibandronate (Boniva), risedronate (Actonel), zoledronic acid (Zometa, Aclasta), cimadronate, clodronate, tiludronate, etidronate, piridronate, and/or functional derivatives and/or analogues and/or esters, and/or salts thereof. In certain embodiments, the nitrogen-containing bisphosphonate preferably comprises zoledronic acid.

In some methods, the dosage of a nitrogen-containing bisphosphonate is from about 30 μg/kg to about 125 μg/kg.

In some embodiments, the nitrogen-containing bisphosphonate is administered prior to the infection. In other embodiments, the nitrogen-containing bisphosphonate is administered at the time of infection. In another embodiment, the nitrogen-containing bisphosphonate is administered after the infection.

In some embodiments, the methods further comprise administering to the subject at least one other therapeutic agent(s) along with the composition and/or pharmaceutical compositions. In some embodiments, the at least one other therapeutic agent is selected from the group consisting of an anti-viral agent, an anti-fungal agent, an anti-bacterial agent, an anti-yeast agent, an anti-parasitic agent, an anti-protozoal agent, an anti-inflammatory agent (e.g., NSAIDS, Cox-2 inhibitors, corticosteroids, etc.), and combinations thereof. In one embodiment, the other therapeutic agent comprises an anti-viral agent. In some embodiments, the anti-viral agent is selected from the group consisting of chloroquine, hydroxychloroquine, remdesivir (Gilead Sciences), favilavir (National Medical Products Administration of China), fusogenix (EntosPharmaceuticals), ChAdOx1 (Univ. of Oxford), Gimsilumab (Roivant Sciences), AdCOVID (Altimmune), TJM2 (I-Mab Biopharma), AT-100 (Airway Therapeutics), TZLS-501 (Tiziana Life Sciences), OYA1 (OyaGen), BPI-002 (BeyondSpring), INO-4800 (Inovio Pharmaceuticals), NP-120 (Algernon Pharmaceuticals), APNO1 (APEIRON Biologics), mRNA-1273 (Moderna), Avian Coronavirus Infectious Bronchitis Virus (IBV) vaccine (MIGAL Research Institute), TNX-800 (Tonix Pharmaceuticals), Btilacidin (Innovation Pharmaceuticals), Recombinant subunit vaccine (Clover Biopharmaceuticals), leronlimab (CytoDyn), Linear DNA Vaccine (Applied DNA Sciences), BXT-25 (BIOXYTRAN), INO-4700 (Inovio), Actemra (Roche), Galidesiver (Biocryst Pharma), REGN3048-3051 (Regeneron), SNG001 (Synairgen Research), Aminoboost (lattice Biologics), and the like.

In some applications, it may be advantageous to utilize the active agent in a “vectorized” form, such as by encapsulation of the bisphosphonate active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

### Examples

Previously Use of NCB to provide analysis of effectiveness of NCB in Patients With COVID-19 (SARS-CoV-2)

Objectives

Determine risk of death after COVID-19 diagnosis associated with prior treatment with nitrogen-containing bisphosphonates

Determine risk of death, ventilator use, or ICU admission associated with prior treatment with nitrogen-containing bisphosphonates

Population

Patient-level data from administrative datamarts maintained by the participating members including STAR CRN, INSIGHT, Capricorn, REACHNet and OneFlorida CRNs will be utilized, identifying patients age≥60 years, with COVID-19 diagnosis. Sites will query for the defined cohort of COVID-19 patients with available data from Jan. 1, 2017 through Sep. 30, 2020. Data structure for protocol development has utilized PCORNet Common Data Model; however, data harmonization may be required.

TABLE 1
Exposure Variable

| Medication | RxNORM code - rxcui |
|---|---|
| Alendronate | 114265, 607553, 1248078, 317541, 316968, 1535727, 46041, |
| | 1007560, 203152, 236083, 904495, 904433, 904399, 904421, |
| | 904465, 1248083, 904427, 904397, 904420, 904426, 904432, |
| | 904463, 904493, 1537103, 904398, 904464, 1537104, 904492, |
| | 1248077, 904396, 904462, 904431, 904425, 904447, 904405, |
| | 904419, 904394, 904404, 904418, 904424, 904430, 904445, |
| | 904395, 904446, 904461, 1536440, 1367006, 1367007, 1367008, |
| | 1367009, 1367010, 1173285, 1173286, 1173287, 1173288, |
| | 1248082, 1151131, 1151133, 1151137 |
| Zoledronic Acid | 285143, 705872, 1649574, 77655, 1546014, 351945, 1114087, |
| | 705875, 1114086, 575698, 705873, 1865671, 1865680, 1114085, |
| | 705824, 351114, 1865665, 1865663, 1114084, 358826, 705823, |
| | 1865664, 1156253, 1178326, 1188174, 1151126 |
| Risedronate | 753143, 1020066, 905101, 905093, 10312, 317541, 905100, |
| | 1020065, 905092, 73056, 607278, 60334, 1356127, 1356128, |
| | 1926958, 905043, 905034, 905026, 905030, 905025, 905029, |
| | 905033, 905042, 753145, 1020064, 905032, 905024, 905028, |
| | 905083, 905041, 905023, 905027, 905031, 905040, 905082, |
| | 1020063, 617278, 1165476, 1165477, 1169918, 1169919, |
| | 1151131, 1151133 |

TABLE 2
Outcome Variables

| Variable | Definition | Data Source |
|---|---|---|
| Death* | Death record present | DEATH |
| Ventilator Use | CPT code 94002 and 94003 OR | PROCEDURE |
| | ICD-10 diagnosis code Z99.11, Z99.89 | DIAGNOSIS |
| | after COVID-19 diagnosis | |
| ICU Admission | CPT code 99291 and 99292 after | PROCEDURE |
| | COVID-19 diagnosis | |

*Death recorded differently by CDRN group

TABLE 3
Independent Variables at time of COVID-19 diagnosis

| Variable | Definition | Data Source |
|---|---|---|
| Age | Difference between [ADMIT_DATE] | ENCOUNTER |
| | at COVID-19 diagnosis | DEMOGRAPHIC |
| | and [BIRTH_DATE], in years | |
| Gender | [SEX] | DEMOGRAPHIC |
| Race | [RACE] | DEMOGRAPHIC |
| Ethnicity | [HISPANIC] | DEMOGRAPHIC |
| Body Mass Index | First [HT] and [WT] recorded | VITAL |
| | during COVID-19 diagnosis, | |
| | earned forward | |
| Tobacco use | [SMOKING] | VITAL |
| Medications | | MED_ADMIN |
| Dexamethasone | | |
| Hydroxychloroquine | | |
| Antibody infusion | | |
| Comorbidities | | CONDITION |
| Diabetes | | |
| Heart disease | | |
| COPD | | |
| Chr kidney dis | | |
| Liver dis | | |
| Laboratories | | LAB_RESULT_CM |

TABLE 4
Independent Variables associated with NCB prescription

| Variable | Definition | Data Source |
|---|---|---|
| Age | [BIRTH_DATE] | DEMOGRAPHIC |
| Gender | [SEX] | DEMOGRAPHIC |
| Race | [RACE] | DEMOGRAPHIC |
| Ethnicity | [HISPANIC] | DEMOGRAPHIC |
| Comorbidities | [CONDITION] | CONDITION |
| Prior fracture | [CONDITION_TYPE] | |
| Rheumatoid arthritis | Presence of | |
| Malabsorption disorder | corresponding diagnosis | |
| Liver disease | code (e.g., ICD-10) on 2 | |
| Diabetes | separate dates | |
| Chr kidney disease | | |
| Cerebrovascular disease | | |
| Cardiovascular disease | | |
| Alcohol abuse | | |
| Tobacco use | [SMOKING] | VITAL |
| Medication | | DISPENSING |
| Corticosteroid use* | | PRESCRIBING |
| Aromatase inhibitor | | |
| Androgen deprivation | | |
| Antiseizure drug | | |

*Prescription equivalent to prednisone 5 mg daily for 3 months

Statistical Analysis

NCB exposure is defined as prescription of relevant NCB within 3 years prior to COVID-19 diagnosis (Table 1). To control for non-random assignment to NCB prescription, the propensity (probability) for prescription is calculated and used as a stratification variable. Generally, the estimate of the propensity is a large model, containing numerous predictors including patient demographics, provider preferences, medical comorbidities, and concurrent medications (Table 4).

Conditional Logistic Regression model is employed to calculate risk of the composite outcome of death, ventilator use, and ICU admission, as well as the risk of death as the primary outcome. The independent variable of interest is NCB prescription (yes/no), as defined above. The propensity for NCB prescription defines the strata for each model. Expected confounding variables include age, gender, race/ethnicity, BMI, diabetes among other underlying medical comorbidities, and medical treatments provided (Table 3). Because of the increasing understanding of COVID-19 disease and treatment, the proposed confounding variables represent the minimum set of variables to be used. The general model can be represented as:

$$logit ( Outcome ) = log \frac{P ( Outcome )}{1 - P ( Outcome )} = {β}_{0} + {β}_{1} ( NCB Prescription ) + \sum {β}_{i} {X}_{i}$$

where Xᵢ represent the confounding independent variables which are thought to relate linearly with the logit(Outcome). Primary analysis is stratified by NCB propensity score quintile; sensitivity analyses using direct matching by propensity score will be performed.

### SUMMARY

statistics (average effect, S.E., 95% CI) across the strata for overall effect will be calculated using standard meta-analytic techniques.

Key Milestones for Participating Sites

Participating sites receive query distributed by PCORnet Coordinating Center or other agreed upon technical team via a secure method (e.g., PopMedNet, Duke Box, SFTP), run the query locally against their PCORnet datamart, and return patient-level, de-identified results to Duke for analysis via the agreed upon secure method. The table below provides target timeframes for task completion:

TABLE 5

| Milestone | Description | Timeframe |
|---|---|---|
| Contracting and IRB | Site executes a contract with Duke | 3-4 weeks |
| submission | and completes local IRB | |
| | submission | |
| Development of data | Code to be developed by PCORnet | 4-6 weeks |
| extraction SAS code to | Coordinating Center or other | |
| generate analytic dataset | programming group. | |
| Analytic Study Query | Sites receive and run analytic study | 4-6 weeks |
| | query and return results through | after |
| | agreed upon secure method. | receiving |
| | | analytic study |
| | | query |

Results

Nitrogen-containing bisphosphonate to Treat Hospitalized Patients With COVID-19 (SARS-CoV-2) with a finding that nitrogen-containing bisphosphonates are associated with lower ventilator use and ICU admission among older adults with COVID-19.

Background: Widely used in the treatment of osteoporosis, nitrogen-containing bisphosphonates (NCBs) have been associated with reduced mortality and cardiopulmonary morbidity among older adults. Prior preclinical studies suggest NCB may mediate inflammation and subsequent fibrosis. Containing on these prior studies, it is hypothesized that NCPs could reduce ventilator use and ICU use admission among COVID-19 patients.

Methods: a retrospective cohort analysis of administrative data was conducted among 57 National Patient-Centered Clinical Research Network studies (PCORNets DataMarts) through September 2020.

Individuals age>50 years with a diagnosis or positive laboratory test for COVID-19 during the study period were identified. Use of nitrogen-containing bisphosphonates (NCB) was defined as a prescription for Alendronate or Zoledronic acid within 3 years prior to the index COVID-19 diagnosis.

The outcome of interest was ventilator use or ICU admission.

Results: Of 62,214 COVID-19 subjects identified during the study period, 1,014 were previously prescribed an NCB. There were 185 subjects who were previously prescribed Zoledronic acid. The mean age of the cohort was 65.1 years, with 51% female. The majority of subjects were White (44%), with 26% Black and 21% Hispanic. Among those prescribed an NCB, there was a decrease in the risk of ventilator use or ICU admission, but the result was not statistically significant. (RR 0.87, 95% CI: 0.72-1.04).

However, In subgroup analysis restricting to subjects age 60-89 years, prior NCB was associated with significant decrease in ventilator use or ICU admission (RR 0.72, 95% CI: 0.59-0.88).

Conclusion: Among older COVID-19 patients between the age of 60 and 89 years, prior exposure to NCB was associated with a 28% reduction in ventilator use and ICU admission. These results suggest a prophylactic role of NCBs in mitigating the severity of COVID-19.

Because many patients hospitalized with COVID-19 infection have significant inflammation in the lungs and heart, thus it is believed, in light of the above data, that patients who have received a course of NCB treatment within the last 3 years will have a 15-25% improvement in their rate of survival after hospitalization compared to patients who have not had exposure to NCB therapy.

Any patents or publications mentioned in this specification are indicative of the levels of those skilled in the art to which the present disclosure pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

The References Cited Herein are Incorporated by Reference Herein for all Purposes.
1. Zheng, Ying-Ying & Ma, Yi-Tong & Zhang, Jin-Ying & Xie, Xiang. (2020). COVID-19 and the cardiovascular system. Nature Reviews Cardiology. 17. 1-2.
2. Luis Ortega-Paz, Davide Capodanno, Gilles Montalescot. Dominick J. Angiolillo; (2020), Coronavirus Disease-2019-Associated Thrombosis and Coagulopathy: Review of the Pathophysiological Characteristics and Implications for Antithrombotic Management, Journal of the American Heart Association, 10(3):e019650.
3. Peter A Kavsak, Ola Hammarsten, Andrew Worster, Stephen W Smith, Fred S Apple, 2021, Cardiac Troponin Testing in Patients with COVID-19: A Strategy for Testing and Reporting Results, Clinical Chemistry, Volume 67, Issue 1, Pages 107-113.
4. Jacqueline R. Centera, Kenneth W. Lyles, Dana Bliuca, 2020. Bisphosphonates and lifespan; Bone, v.141, 115566.

1. A method of treating and/or reducing the negative effects of an infection-induced cardiomyopathy in a subject, the method comprising administering to the subject a therapeutically effective amount of a nitrogen-containing bisphosphonate or a pharmaceutically effective salt thereof.

2. The method according to claim 1, wherein the negative effects of the infection-induced cardiomyopathy are selected from the group consisting of ACE2-mediated direct damage, hypoxia-induced myocardial injury, increased levels of D-dimers causing coagulopathy, cardiac microvascular damage, cytokine release syndrome and increased mortality in the subject.

3. The method according to claim 2, wherein the nitrogen-containing bisphosphonate is selected from the group consisting of pamidronate (APD, Aredia), neridronate (Nerixia), olpadronate, alendronate (Fosamax), ibandronate (Boniva), risedronate (Actonel), zoledronic acid (Zometa, Aclasta), cimadronate, clodronate, tiludronate, etidronate, piridronate, and/or functional derivatives and/or analogues and/or esters, and/or salts thereof.

4. The method according to claim 3, wherein the nitrogen-containing bisphosphonate comprises zoledronic acid.

5. The method according to claim 1, wherein the cardiomyopathy is due to an infection selected from the group consisting of a viral infection, a yeast infection, a bacterial infection, a parasitic infection, a fungal infection, and combinations thereof.

6. The method according to claim 5, wherein the infection comprises a viral infection.

7. The method according to claim 6, wherein the viral infection is caused by a coronavirus.

8. The method according to claim 7, wherein the viral infection is selected from the group consisting of SARS, MERS, and COVID-19.

9. The method according to claim 8, wherein the viral infection comprises COVID 19 or a variant thereof.

10. The method according to claim 1, wherein the dosage of nitrogen-containing bisphosphonate is from about 30 μg/kg to about 125 μg/kg.

11. The method according to claim 1, wherein the nitrogen-containing bisphosphonate is administered prior to the subject being infected with the infection, at the time when the subject is infected or after the subject is infected with the infection.

12. The method according to claim 1, the method further comprising administering to the subject at least one other therapeutic agent(s) selected from the group consisting of an anti-viral agent, an anti-fungal agent, an anti-bacterial agent, an anti-yeast agent, an anti-parasitic agent, an anti-protozoal agent, an anti-inflammatory agent and combinations thereof.

13. The method according to claim 12, wherein the at least one other therapeutic agent comprises an anti-viral agent.

14. The method according to claim 13, wherein the anti-viral agent is for the treatment of a coronavirus.

15. The method according to claim 14 in which the anti-viral agent is for the treatment of COVID-19 or variant thereof.

16. The method according to claim 15, wherein the anti-viral agent is selected from the group consisting of chloroquine, hydroxychloroquine, remdesivir (Gilead Sciences), favilavir (National Medical Products Administration of China), fusogenix (EntosPharmaceuticals), ChAdOx1 (Univ. of Oxford), Gimsilumab (Roivant Sciences), AdCOVID (Altimmune), TJM2 (I-Mab Biopharma), AT-100 (Airway Therapeutics), TZLS-501 (Tiziana Life Sciences), OYA1 (OyaGen), BPI-002 (BeyondSpring), INO-4800 (Inovio Pharmaceuticals), NP-120 (Algernon Pharmaceuticals), APNO1 (APEIRON Biologics), mRNA-1273 (Moderna), Avian Coronavirus Infectious Bronchitis Virus (IBV) vaccine (MIGAL Research Institute), TNX-800 (Tonix Pharmaceuticals), Btilacidin (Innovation Pharmaceuticals), Recombinant subunit vaccine (Clover Biopharmaceuticals), leronlimab (CytoDyn), Linear DNA Vaccine (Applied DNA Sciences), BXT-25 (BIOXYTRAN), INO-4700 (Inovio), Actemra (Roche), Galidesiver (Biocryst Pharma), REGN3048-3051 (Regeneron), SNG001 (Synairgen Research), Aminoboost (lattice Biologics), and combinations thereof.

17. The method according to claim 12, wherein the at least one other therapeutic agent comprises an anti-inflammatory agent.

18. The method according to claim 17, wherein the anti-inflammatory agent is selected from the group consisting of an NSAID, Cox-2 inhibitor, corticosteroids, and combinations thereof.

19. The method according to claim 12, wherein the at least one other therapeutic agent(s) is administered prior to the nitrogen-containing bisphosphonate, with the nitrogen-containing bisphosphonate or after delivery of the nitrogen-containing bisphosphonate.

20. The method according to claim 4, further comprises administering to the subject a therapeutically effective amount of Vitamin D.

21. The method according to claim 20, wherein the nitrogen-containing bisphosphonate is administered before, simultaneously or after the dosage of Vitamin D.

22. The method according to claim 21, wherein the nitrogen-containing bisphosphonate is administered transdermally, orally or intravenously.

23. A composition comprising a nitrogen-containing bisphosphonate in a therapeutically effect amount to treat an infection-induced cardiomyopathy in a subject, the composition is formulated to deliver from about 30 μg/kg to about 125 μg/kg of the nitrogen-containing bisphosphonate to the subject.

24. The composition according to claim 23, wherein the nitrogen-containing bisphosphonate comprises zoledronic acid.

25. The composition according to claim 24, wherein the amount of zoledronic acid comprises about 100 μg/kg.

26. A pharmaceutical composition for the treatment of an infection-induced cardiomyopathy in a subject, the composition comprising a nitrogen-containing bisphosphonate in a therapeutically effect amount to treat an infection-induced cardiomyopathy in a subject, the composition is formulated to deliver from about 30 μg/kg to about 125 μg/kg of the nitrogen-containing bisphosphonate to the subject and a pharmaceutically acceptable carrier.

27. The pharmaceutical composition according to claim 26, whereon the nitrogen-containing bisphosphonate comprises zoledronic acid.

28. The pharmaceutical composition according to claim 27, wherein the amount of zoledronic acid comprises about 100 μg/kg.

29. (canceled)
